(19) United States
(12) Patent Application Publication
(10) Pub. No.: US 2015/0157390 A1
(43) Pub. Date: Jun. 11, 2015

(54) HAND-HELD SMOKE EVACUATION INSTRUMENT

(71) Applicant: Richard P. Fleenor, Greenwood Village, CO (US)

(72) Inventor: Richard P. Fleenor, Greenwood Village, CO (US)

(21) Appl. No.: 14/561,082

(22) Filed: Dec. 4, 2014

Related U.S. Application Data
(60) Provisional application No. 61/911,864, filed on Dec. 4, 2013.

(51) Int. Cl. A61B 18/14; A61B 18/12; A61B 19/00

(52) U.S. Cl. CPC A61B 18/14; A61B 19/5202; A61B 18/1206; A61B 2218/008

(57) ABSTRACT

A hand-held instrument is provided for smoke evacuation at a tissue site. The instrument may include a housing sized for hand-held use and defining at least a portion of an internal volume of the instrument. An inlet port and an outlet port are provided for gas passage in to and out of the internal volume, wherein an impeller is disposed within the internal volume between the inlet port and the outlet port. In some embodiments, an on-board motor may be disposed within the internal volume to rotate the impeller and induce gas flow in to the inlet port and out of the outlet port. A filter may be located at the outlet port for filtering gas flowing out of the outlet port. In some embodiments, an energy emission component (e.g., an electrosurgical electrode) may be integrated into the hand-held instrument. Additionally, some embodiments may include one or a plurality of light emitters for emitting light in a direction distal to a distal end of the hand-held instrument.

### RELATED APPLICATIONS

This application claims priority to U.S. Provisional Application No.: 61/911,864 filed Dec. 4, 2013, entitled “HAND-HELD SMOKE EVACUATION INSTRUMENT,” which is incorporated herein by reference in its entirety.

### FIELD OF THE INVENTION

The present invention relates to a hand-held instrument for smoke evacuation, and more particularly to a medical-related instrument for evacuation of smoke at a patient tissue site at which a medical procedure is conducted via the application of energy to the tissue site.

### BACKGROUND OF THE INVENTION

Numerous medical procedures entail the application of energy to a patient tissue site. By way of primary example, electrosurgical procedures entail the application of an electrosurgical signal to cut tissue and/or coagulate tissue. In conjunction with such procedures, a volume of undesirable smoke may be generated. The smoke may obscure a tissue site, present an offensive odor, and otherwise contain potentially harmful constituents to those exposed to it.

### SUMMARY OF THE INVENTION

To address one or more of the above-referenced problems, a hand-held instrument is provided for smoke evacuation. The instrument may be conveniently utilized in connection with a medical procedure in which electrical or another form of energy is applied to a tissue site, thereby resulting in the generation of smoke.

In one embodiment, a hand-held instrument includes a housing sized for hand-held use and defining at least a portion of an internal volume of the hand-held instrument. The instrument may further include an inlet port and an outlet port for gas passage into and out of the internal volume. An impeller may be disposed within the internal volume between the inlet port and the outlet port. To rotate the impeller, a motor may be disposed within the internal volume, wherein upon powered rotation of the impeller by the motor, gas flow into the inlet port and out of the outlet port may be induced. For example, an internal gas flow rate of at least 3.0 ft.³/min. may be provided. As may be appreciated, the provision of a hand-held instrument having on-board componentry for powered smoke evacuation through the instrument yields an effective and efficient solution to one or more of the problems noted hereinabove, and is particularly apt for extracorporeal use.

In some implementations, the hand-held instrument may comprise a filter located at the outlet port for filtering smoke-laden gas flowing in to the inlet port and out of the outlet port. The filter may be provided for capture of particles, e.g., particles having a minimum size, or cross-dimension, of 0.12 microns or larger. Further, the filter may provide for adsorption and/or absorption of undesirable gas smoke constituents, e.g., cytotoxins, papillomavirus, mutagens. Optionally, the filter may include a filter element that is readily replaceable.

In some embodiments, the hand-held instrument may be provided so that the inlet port is located at or proximate to a distal end of the hand-held instrument, and wherein the outlet port is located proximal to the impeller. To provide for enhanced gas flow from the impeller to the outlet port, the hand-held instrument may include a gas flow member located proximal to the impeller within the internal volume between the impeller and the outlet port. The gas flow member may comprise a plurality of blades extending in a direction parallel to a desired gas flow path so as to direct the gas flow from the impeller to the outlet port.

In one approach, the outlet port may be disposed along a side of the housing of the hand-held instrument. For example, the outlet port may be located along an elongate side portion of the housing so as to direct gas flow from the outlet port laterally away from the hand-held instrument. In some embodiments, the hand-held instrument may further include an internal side wall that defines a portion of the internal volume proximal to the impeller, wherein a portion of the side wall is angled (e.g., curved) across a longitudinal axis of the hand-held instrument to the outlet port so as to direct gas flow from the impeller to the outlet port.

In certain embodiments, the housing of the hand-held instrument may include a first member that includes the outlet port, and a second member that includes the inlet port. The first member and the second member may be interconnected and otherwise disposed for relative movement therebetween. For example, the first and second members may be disposed for relative movement therebetween along a longitudinal axis of the hand-held instrument, wherein the second member is positionable in at least a retracted first position and an extended second position distal to the first position. In contemplated approaches, the second member may be selectively positionable by a user relative to the first member at a continuum of positions along the longitudinal axis. For such purposes, the first and second members may be slidably interconnected along the longitudinal axis. In that regard, a proximal end portion of the second member may be provided so as to extend into a distal end portion of the first member for telescoping movement relative thereto. In such embodiments, the first member and second member may each be of a tubular configuration.

In some implementations, the hand-held instrument may also include at least one light emitter for emitting light in a direction distal to a distal end of the housing. For example, in embodiments in which the housing includes a first member and a second member, as described above, the light emitter(s) may be supportably interconnected to and outside of the second member for emitting light in a direction distal to a distal end of the second member. In some implementations, a plurality of light emitters may be supportably interconnected to the housing. For example, at least three light emitters may be interconnected in spaced relation about the housing.

In some embodiments, at least one hand switch may be included in the hand-held instrument for operational control of the motor. For example, the hand switch may be provided for selectively turning the motor on and off, and in some implementations to control the speed of the motor output and resulting impeller rotation and attendant gas flow rate. In some implementations, the hand switch(es) may be conveniently located along a side of the housing, wherein a user may grasp/manipulate the hand-held instrument with a given hand so as to orient the inlet port where desired for smoke intake, and also utilize the same hand to operate the hand switch. Where the housing comprises first and second members, as discussed above, the hand switches may be conveniently located on the proximal first member, distal to the outlet port.

In some implementations, the motor may be disposed proximal to the impeller and the outlet port. Such positioning facilitates the maintenance of an open internal volume between the inlet port and the outlet port so as to optimize gas flow therebetween. Further, such positioning facilitates interconnection of the motor with a power source. In one approach, the hand-held instrument may comprise at least one battery for electrically powering the motor, wherein both the motor and the battery may be disposed within the internal volume proximal to the impeller and the outlet port. In some implementations, the one or more batteries may be replaceable. In another approach, the electrosurgical instrument may be provided with or interconnectable to electrical cabling at a proximal end, wherein the electrical cabling may be electrically interconnected or interconnectable to the motor. The electrical cabling may extend proximally away from the housing to a proximal end that may be electrically interconnectable to an appropriate electrical power source (e.g., via a plug-in coupler end), or that may be electrically interconnected to one or a plurality of batteries located in a battery module. In the later regard, a battery module may be provided along the electrical cabling for supporting at least one removable/replaceable battery in electrical contact with the electrical cabling at a location that is advantageously spaced from the housing and components housed therein.

In an embodiment, a hand-held instrument may include a housing sized for hand-held use that defines at least a portion of an internal volume of a hand-held instrument, an inlet port and an outlet port for gas passage into and out of the internal volume, and an impeller disposed within the internal volume between the inlet port and the outlet port. Additionally, an energy emission component may be integrated for energy application to a tissue site. In contemplated approaches, the energy emission component may be supportably connected to and extend distally from the hand-held instrument at a distal end of the housing. Advantageously, the energy emission component may be disposed in parallel or aligned relation to a longitudinal axis (e.g., a center axis) of the housing at the distal end thereof. The energy emission component may comprise an electrosurgical electrode, a laser beam emitter, or another component for conveying energy to a tissue site to achieve a desired effect (e.g., to cut and/or coagulate tissue).

In one arrangement, the hand-held instrument may further include at least one light emitter supportably interconnected to the housing for emitting light in a direction distal to a distal end of the housing, e.g. so as to illuminate a volume that includes all or at least a distal end portion of the energy emission component (e.g. electrosurgical electrode) and/or tissue site. For example, a plurality of light emitters (e.g. light emitting diodes) may be provided in spaced relation about the housing (e.g. in a ring-shaped arrangement at a common location along a center axis of the housing), wherein the light emitters may illuminate a volume that includes at least a distal end portion of an electrosurgical electrode and/or a tissue site.

In contemplated embodiments in which the housing comprises a first member and a second member as described above, an energy emission component (e.g., an electrosurgical electrode) may be supportably interconnected to and extend distally away from the distal end of the second member for co-movement therewith. In turn, a user may selectively position the second member and interconnected energy emission component at a desired position relative to the first member via selective sliding movement of the second member relative to the first member. For example, the first and second members may be slidably interconnected as described above so that the second member may be selectively positioned by a user at a continuum of positions along a longitudinal axis of the hand-held instrument. In such embodiments that integrate an electrosurgical electrode, the electrode may be disposed in parallel or aligned relation to the longitudinal axis. Further, the instrument may include a first electrical contact (e.g., an elongate metal strip) mounted to the first member (e.g., parallel to a longitudinal axis of the instrument) and a second electrical contact (e.g., a metal strip) mounted to the second member so that the second electrical contact is maintained in slidable contact engagement with the first electrical contact when the second member is selectively positioned by a user at any of the continuum of positions.

In some embodiments the hand-held instrument may include cabling interconnected to an integrated energy emission component and extending proximally from the housing, wherein the cabling may be interconnected to a source for providing an energy signal to the energy emission component. For example, electrical cabling may be provided for electrical interconnection to an electrosurgical generator, wherein the electrosurgical generator may provide an electrosurgical tissue cutting signal and/or an electrosurgical tissue coagulation signal to an integrated electrosurgical electrode comprising the hand-held instrument upon user manipulation of one or more control switches provided on the housing of the hand-held instrument.

In various embodiments, a motor may be included in the integrated hand-held instrument, as described above. Further, the hand-held instrument may include an onboard power source (e.g., one or more batteries that may be replaceable) or electrical cabling electrically interconnectable to an appropriate source for powering operation of the motor for smoke evacuation at a tissue site. In one approach, electrical cabling may be interconnected to the motor and routed together with electrical cabling for interconnection to an electrosurgical generator, wherein a proximal coupler end may comprise one or more batteries (e.g., replaceable DC batteries) for powering the motor.

In conjunction with a hand-held instrument for smoke evacuation, an inventive method is also provided. In one embodiment, the method may include the steps of utilizing a motor of a hand-held instrument to induce gas flow into an inlet port and out of an outlet port of a hand-held instrument, and positioning the hand-held instrument so as to locate the inlet port of the hand-held instrument proximate to a smoke source (e.g., a patient tissue site), wherein smoke-laden gas is drawn into the inlet port of and evacuated through the outlet port of the hand-held instrument. The method may further include a step of filtering the gas flow, e.g., by interconnecting a filter member to the hand-held instrument at the outlet port. In some embodiments, the method may further provide for directing the gas flow laterally away from the hand-held instrument through the outlet port thereof.

In certain implementations, the motor utilization step may include controlling the initialization and/or termination of operation of the motor via manipulation of at least one hand switch of the hand-held instrument. Such motor control may be achieved by the same user hand that is also employed for positioning the hand-held instrument.

The method may further include the step of directing gas flow through at least a portion of the internal volume of the hand-held instrument along the longitudinal axis of the hand-held instrument. By way of example, a gas flow member may be disposed within an internal volume of the hand-held instrument between an impeller and the outlet port to direct the gas flow therebetween.

In some embodiments, the method may further include the step of supplying a signal to an energy emission component of the hand-held instrument. As noted, the energy emission component may comprise an electrosurgical electrode, wherein an electrical signal may be supplied for tissue cutting and/or tissue coagulation. When employed, an energy emission component may be supportably interconnected to and extend distally from a distal end of a housing of the hand-held instrument. In that regard, the housing may include a first member that includes the outlet port and a second member that includes the inlet port, wherein the first member and second member are slidably interconnected and disposed for relative movement therebetween. In such arrangements, the energy emission component may be supportably connected to the second member for co-movement therewith wherein the method may further include the step of locating the energy emission component by slidably positioning the second member relative to the first member.

In embodiments employing an energy emission component, the energy emission component may be employed to deliver energy to a patient tissue site in conjunction with a medical procedure. In turn, such application of energy may result in the generation of smoke at the patient tissue site. As may be appreciated, in such embodiments the method provides for the convenient single-hand positioning and use of a single hand-held instrument for both the application of energy to a patient tissue site and for the removal of smoke generated in connection with the energy application.

Numerous additional features and advantages of the present invention will become apparent to those skilled in the art upon consideration of the embodiment descriptions provided hereinbelow.

### BRIEF DESCRIPTION OF THE DRAWINGS

FIG. 1A is a top view of a hand-held instrument embodiment.

FIG. 1B is a side view of the hand-held instrument embodiment of FIG. 1A.

FIG. 1C is a bottom view of the hand-held instrument embodiment of FIG. 1A.

FIG. 1D is a distal end view of the hand-held instrument embodiment of FIG. 1A.

FIG. 2A is a cross-sectional side view of the hand-held instrument embodiment of FIG. 1A.

FIG. 2B is a cross-sectional top view of the hand-held instrument embodiment of FIG. 1A.

FIG. 3 is an exploded view of components of the hand-held instrument embodiment of FIG. 1A.

FIG. 4 is a perspective view of the hand-held instrument embodiment of FIG. 1, integrated with electrosurgical componentry in an electrosurgical application.

FIG. 5 is a perspective view of a modified version of the hand-held instrument embodiment of FIG. 4.

FIG. 6 is a perspective view of the hand-held instrument embodiment of FIG. 5, supplemented to include light emitters.

FIG. 7 is a schematic illustration of hand-held instrument showing electrical connections of powered components.

### DETAILED DESCRIPTION

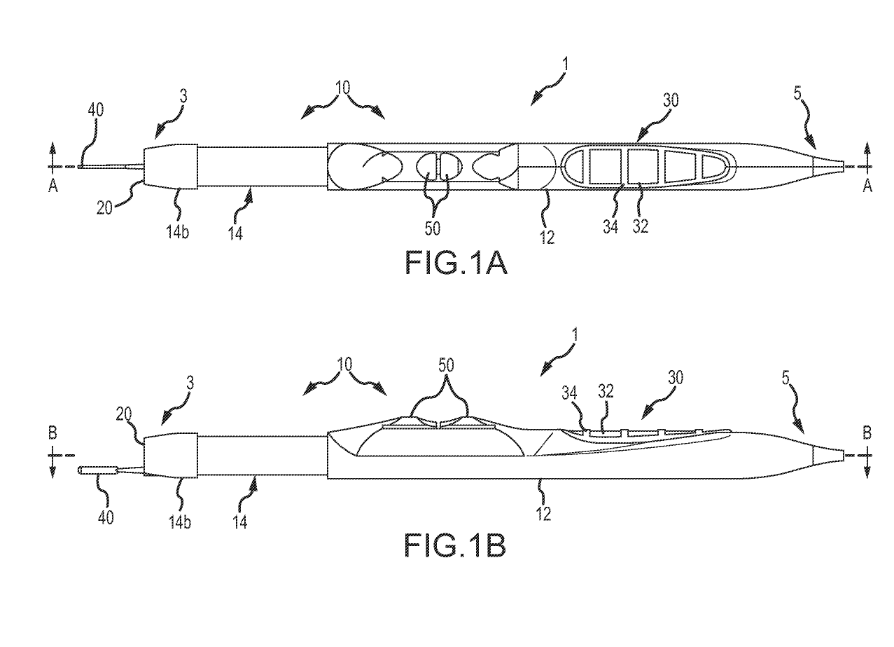

FIGS. 1A, 1B, 1C, and 1D illustrate various views of one embodiment of a hand-held instrument 1 intended for medical-related use. By way of primary example, hand-held instrument 1 may be utilized for smoke evacuation of a patient tissue site at which a medical procedure is conducted via the application of energy to the tissue site. As may be appreciated, energy application may generate smoke which may obscure the tissue site and/or otherwise result in undesirable environmental conditions for medical personnel and the patient. Additional applications and modifications of the hand-held instrument 1 will become apparent upon further description.

Hand-held instrument 1 may include a housing 10 sized for hand-held use and defining a portion or all of an internal volume. In the illustrated embodiment, housing 10 is of an elongate and cylindrical configuration to facilitate single hand manipulation by a user. The hand-held instrument 1 includes a proximal end 3 and a distal end 5. A gas inlet port 20 is provided proximate to the distal end 3, and a gas outlet port 30 is provided along an outer side wall of housing 10, proximal to the inlet port 20. As will be further described, in operation, smoke-laden gas may be drawn in to inlet port 20, pass through an internal volume of hand-held instrument 1, and exhausted through outlet port 30 to improve visual observation of a tissue site. Further, outlet port 30 may be provided with smoke filtration functionality to reduce undesirable airborne smoke constituents, thereby improving environmental conditions at a medical procedure site.

In the illustrated embodiment, housing 10 may comprise a first member 12 that includes the outlet port 30, and a second member 14 that includes the inlet port 20. The first member 12 may be sized and contoured for single hand manipulation of the hand-held instrument 1 by a user, wherein inlet port 20 may be positioned where desired relative to a smoke source (e.g., at a surgical tissue site). As shown, first member 12 and second member 14 may have cylindrical outer configurations.

The first member 12 and the second member 14 may be interconnected and disposed for relative movement therebetween, thereby facilitating user selected positioning of the inlet port 20 relative to the first member 12. For example, the first member 12 and second member 14 may be provided for relative movement therebetween along a longitudinal axis of the hand-held instrument 1. FIGS. 1A and 1B illustrate the second member 14 selectively positioned in an extended first position relative to first member 12. As shown in FIG. 1C, the second member 14 is selectively positioned in a retracted second position relative to first member 12. Such relative positioning may be provided by slidably positioning a proximal end portion of second member 14 within a distal end portion of the first member 12, wherein the second member 14 may be selectively and advantageously positioned at any one of a continuum of positions selected by a user.

As will be further described, hand-held instrument 1 may include internal componentry for inducing gas flow in to inlet port 20, through an internal volume of hand-held instrument 1, and out of outlet port 30. In particular, and as shown in FIG. 1D, an impeller 60 may be disposed within housing 10 between inlet port 20 and outlet port 30 and rotatively powered by an on-board motor (not shown) to draw gas in to inlet port 20. One or more hand control switches 50 may be provided on housing 10 for user control of the motor, e.g., to start and stop motor operation. Such hand control switches 50 may be conveniently located on a side wall portion of first member 12.

The hand-held instrument 1 may be optionally provided to include an integrated energy emission component. Such energy emission component may be provided for use in energy application to a patient tissue site in connection with a medical procedure. By way of example, the energy emission component may comprise one of the following: an electrosurgical electrode, a laser beam emitter (e.g., for ultrasonic tissue cutting and/or tissue sealing).

In the illustrated embodiment, hand-held instrument 1 optionally includes an electrosurgical electrode 40 supportably interconnected to housing 10 at the distal end 3 thereof. For example, the electrosurgical electrode may be interconnected to second member 14 for co-movement therewith. As illustrated, the electrosurgical electrode 40 may be provided to extend distally beyond the inlet port 20, wherein during use the electrosurgical electrode 40 may apply an electrosurgical signal to a tissue site and hand-held instrument 1 may conveniently provide for evacuation of smoke generated in conjunction therewith. In that regard, electrosurgical electrode 40 may be disposed parallel to a longitudinal axis of and gas-flow path through the hand-held instrument 1, thereby facilitating contemporaneous desired positioning of hand-held instrument 1 for both energy application and smoke evacuation.

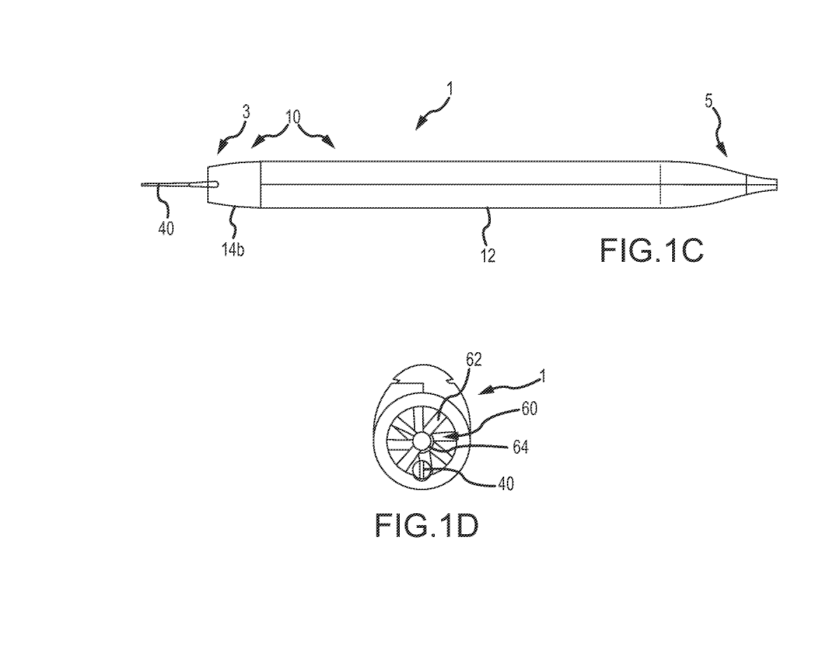

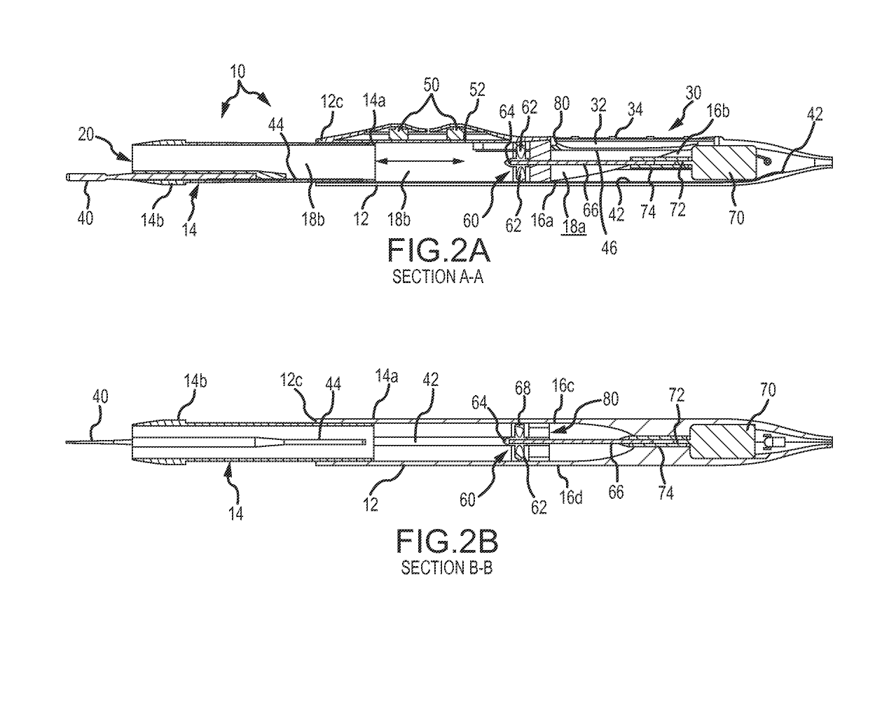

Reference is now made to FIGS. 2A, 2B, and 3 which illustrate internal components of hand-held instrument 1. Such components may include the impeller 60 and a motor 70 operatively interconnected with the impeller 60 to rotate the impeller 60. The impeller 60 may include a plurality of blades 62 extending outward from and spaced about a hub 64, wherein the blades may comprise angled surface portions that extend laterally across an internal volume of the hand-held instrument so as to draw gas into inlet port 20 upon rotation. Hub 64 may be interconnected to a shaft 66 that may extend proximally and interconnect with output shaft 72 of motor 70 via a coupler 74. The impeller 60 may be supportably disposed within an outer ring member 68 that is fixedly positioned relative to an internal surface of first member 12.

In the illustrated embodiment, housing 10 defines an internal volume comprising a first portion 18a located on a proximal side of impeller 60 and a second portion 18b located on a distal side of impeller 60. As shown, the second portion 18b of the internal volume may be cylindrical and may be combinatively defined by the first member 12 and the second member 14. The first portion 18a may be defined by first member 12 and configured to facilitate gas flow from impeller 60 to outlet port 30.

In that regard, and as shown in FIG. 2A, the first member 12 may be provided with an internal surface that angles (e.g., along a smooth curve) from a first side portion 16a to a proximally and radially offset second side portion 16b to define an angled, proximal end wall of internal volume 82, wherein gas flow from impeller 60 to outlet port 30 is enhanced. In the illustrated embodiment, second side portion 16b is proximally offset and radially offset (e.g., 180° relative to a longitudinal axis of hand-held instrument 1) from first side portion 16a. As shown in FIG. 2B, first member 12 may be also provided with internal surfaces that angle (e.g., along smooth curves) from both a third side portion 16c and radially offset fourth side portion 16d proximally toward a longitudinal axis of hand-held instrument 1 to further enhance gas flow from impeller 60 to outlet port 30. As shown, third side portion 16c and fourth side portion 16d may be radially offset (e.g., 180° relative to a longitudinal axis of hand-held instrument 1), and third side portion 16c and fourth side portion 16d may each be radially offset 90° relative to first side portion 16a and second side portion 16b. As described, the first member 12 may be provided with internal surfaces that collectively define a portion of an elliptic parabaloid.

Hand-held instrument 1 may further include a flow path member 80 for directing gas flow, i.e., to enhance laminar gas flow, proximal to impeller 60. The flow path member 80 may comprise a plurality of spaced vanes 82 having surfaces that extend proximally along and in corresponding directions parallel to a longitudinal axis of the hand-held instrument 1 from a distal end of the flow member 80 to a proximal end thereof. The surfaces of vanes 82 function to direct gas flow along the longitudinal axis of the hand-held instrument 1. As shown, the vanes 82 may extend outward from and may be spaced about a hub 84 through which output shaft 72 may rotatably extend. The flow path member 80 may include an outer ring 86 that is fixedly positioned relative to an internal surface of first member 12. In that regard, in addition to enhancing laminar gas flow, the flow path member 82 may also act as a bearing surface to provide support and stability for motor output shaft 72.

As noted above, outlet port 30 may be provided with gas filtering functionality. In that regard, a gas permeable, filter element 32 may be provided with a filter frame 34 across an opening on a side wall portion of first member 12. The filter element 32 may be pleated to increase the filter surface area. By way of example, the filter element 32 may be a hepa filter of fiberglass and/or paper construction. In some embodiments, the filter element 32 may be provided to filter particles having a minimum cross-dimension of 0.12 micron or larger. The filter frame 34 may be provided for snap-fit engagement with a side wall portion of first member 12 surrounding the opening therethrough. The filter frame 34 may be removable/replaceable to permit replacement of filter element 32.

In various implementations, the motor 70 may comprise a brushless DC motor or brushed DC motor. As illustrated in FIGS. 2A and 2B, the motor 70 may be located within first member 12 at a distal end thereof. For example, and as best shown in FIG. 2B, the first member 12 may be internally configured to define an internal pocket and channel sized to retainably receive motor 70 and output shaft 72, respectively. Further, the first member 12 may be internally configured to provide the first portion 82 having the surface configuration described above. To provide such internal configurations, first member 12 may be of a molded, two-part construction, wherein a molded first side member 12a and molded second side member 12b may be configured as shown in FIG. 3 and adjoined after positioning of the various described internal components therein. The second member 14 may be of a molded, tubular construction, wherein a proximal end of the second member 14 may be positioned between the two members 12a, 12b of the first member 12 prior to their interconnection. As illustrated, the first member 12 may be provided with an inward-projecting flange 12c at a distal end and the second member 14 may be provided with an outward projecting flange 14a at a proximal end to restrict removal of the second member 14 from within the distal end of the first member 12. Such flanges 12c and 14a may also be sized to permit sliding movement of second member 14 relative to first member 12, while providing for sufficient engagement so as to maintain a selected relative position of first member 12 and second member 14. Further, the second member 14 may be provided with an enlarged, outward projecting distal end 14b to restrict distal travel of the second member 14 relative to the first member 12.

As noted above, one or more switches 50 (e.g., push or toggle switches) may be provided to control operation of motor 70. In that regard, one or more switches 50 may be located within a protective encasement (e.g., having an elastomeric outer layer). The switches may be electrically interconnected to a circuit board 52 that is electrically connected via electrical connection lines to motor 70.

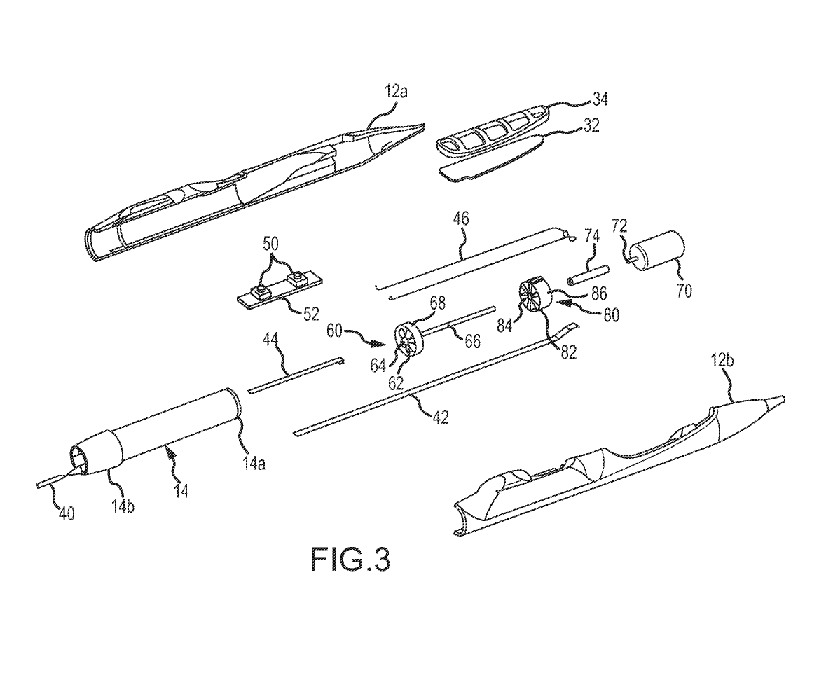

As further noted above, hand-held instrument 1 may optionally include an energy emission component, and in embodiments shown herein, an optional electrosurgical electrode 40 is included. In that regard, reference is now also made to FIG. 4 which illustrates hand-held instrument 1, wherein electrosurgical electrode 40 is electrically interconnected to electrical cabling 90 that extends proximally from the proximal end 3 of hand-held instrument 1 for interconnection to a standard electrosurgical generator via coupler end 92. The electrical cabling 90 and coupler end 92 may be provided for selection and transmission of electrosurgical tissue cutting and tissue coagulation signals from a standard electrosurgical generator. For signal selection, the electrical cabling 90 may be electrically interconnected within hand-held instrument 1 via one or more conductor member 46 and circuit board 52 to switches 50, wherein one of the switches 50 may be utilized to selectively initiate/terminate the provision of an electrosurgical tissue cutting signal to electrode 40, and wherein another one of the switches 50 may be utilized to selectively initiate/terminate the provision of an electrosurgical tissue coagulation signal to electrode 40. Electrical interconnections within the hand-held instrument 1 may be provided so that, upon the provision of an electrosurgical tissue cutting signal or the provision of an electrosurgical tissue coagulation signal, operation of motor 70 is also initiated. Correspondingly, the electrical componentry may be provided so that upon termination of the provision of an electrosurgical tissue cutting signal or termination of an electrosurgical tissue coagulation signal, operation of the motor 70 may terminate. To provide power to the motor 70, separate electrical cabling 100 may proximally extend from the proximal end 3 of hand-held instrument 1 for separate interconnection to an appropriate energy source via plug-in coupler 102. Alternatively, hand-held instrument 1 may include one or more batteries to power motor 70, or in another arrangement electrical cabling 100 may be routed with electrical cabling 90, wherein coupler end 92 may include one or more batteries (e.g., replaceable batteries) for powering motor 70.

In contemplated implementations, the hand-held instrument 1 may provide for gas flow in to inlet port 20 and out of outlet port 30 at a rate of at least 3.0 ft.³/min. For such purposes, second portion 18b of the internal volume may have a diameter of at least 0.5 in. along the length thereof and motor 70 may be provided to rotate impeller 60 at a rate of at least 15,000 revolutions per minute, so as to yield an internal gas flow speed of at least 150 ft./min.

As noted above, electrosurgical electrode 40 may be electrically interconnected to electrical cabling 90. In that regard, and with reference to FIGS. 2A, 2B, and 3, such electrical interconnection may be provided by a first electrical contact 42 and a second electrical contact 44. The first electrical contact 42 may be in the form of an elongate metal strip mounted to an internal surface of the first member 12 so as to extend from a proximal end portion thereof to a distal end portion thereof (e.g., parallel to a longitudinal axis of interfacing portions of first member 12 and second member 14). The second electrical contact 44 may be in the form of a metal strip mounted to an internal surface of second member 14 and extend from a proximal end portion thereof to a contact engagement interface with electrode 40. The second electrical contact 44 may be provided so as to slidably engage the first electrical contact member 42, wherein upon selective positioning of second member 14 relative to first member 12 by a user, the second electrical contact member moves in tandem with the second member 14 while maintaining contact engagement with the first electrical contact 42.

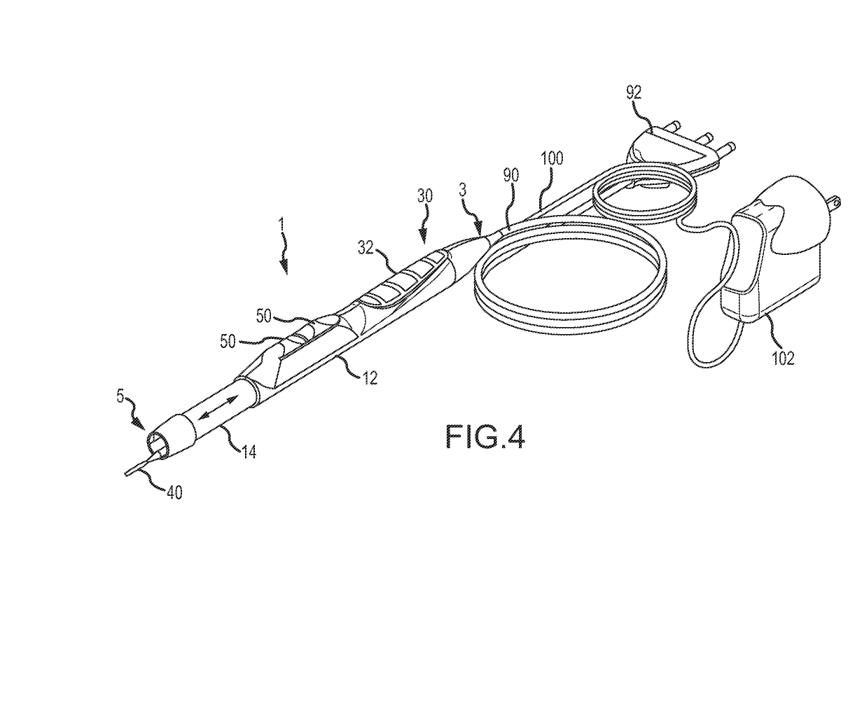

Reference is now made to FIG. 5 which illustrates certain modifications to the hand-held instrument 1 illustrated in FIG. 4. In the modified embodiment, first member 12 of housing 10 may include a bottom channel portion 17 at a distal end for slidably receiving a proximal end portion of a mount 18 that is provided on second member 14 of housing 10. The mount 18 may be provided to support electrosurgical electrode 40 below the inlet port 20 to facilitate laminar gas passage through the internal volume of housing 10.

As described above, the second member 14 may be selectively advanced and retracted relative to the first member 12 along the longitudinal axis of the hand-held instrument 1. Such relative positioning allows the second member 14 and electrosurgical electrode 40 supported thereby to be selectively and advantageously positioned at any one of a continuum positions selected by a user. In the arrangement of FIG. 5, the electrode 40 may extend through mount 18 and engage second electrical contact 44 (not shown) that extends along and is supported by the second member 14, as described above. In turn, a proximal end of second electrical contact 44 may slidably engage the first electrical contact 42 (not shown) that extends along and is supported by the first member 12 as described above.

In another modification to the hand-held instrument shown in FIG. 4, the hand-held instrument of FIG. 5 provides for routing of electrical cabling 90 (not shown) and electrical cabling 100 (not shown) within a common insulative sheath 110 that extends from a proximal end of the hand-held instrument 1 to a distal end that includes coupler end 92 and an adjoined battery module 120. The coupler end 92 may be provided to interface with (e.g. plug in to) a standard electrosurgical generator for the transmission of electrosurgical tissue cutting and tissue coagulation signals via electrical cabling 90, as described hereinabove.

The battery module 120 may be provided for supporting one or more batteries (e.g. two batteries shown in FIG. 5) for providing electrical power (e.g. a 6.0 volt DC signal) to power operation of the motor 70 via electrical cabling 100. The battery module 120 may be provided to facilitate ready placement and replacement of the batteries. For example, as shown in FIG. 5 the battery module 120 may include tubular supports 122 and for slidable placement of batteries thereinto and slidable removal of batteries therefrom. The provision of battery module 122 at a location spaced from the housing 10 of electrosurgical instrument facilitates compliance with sterilization requirements.

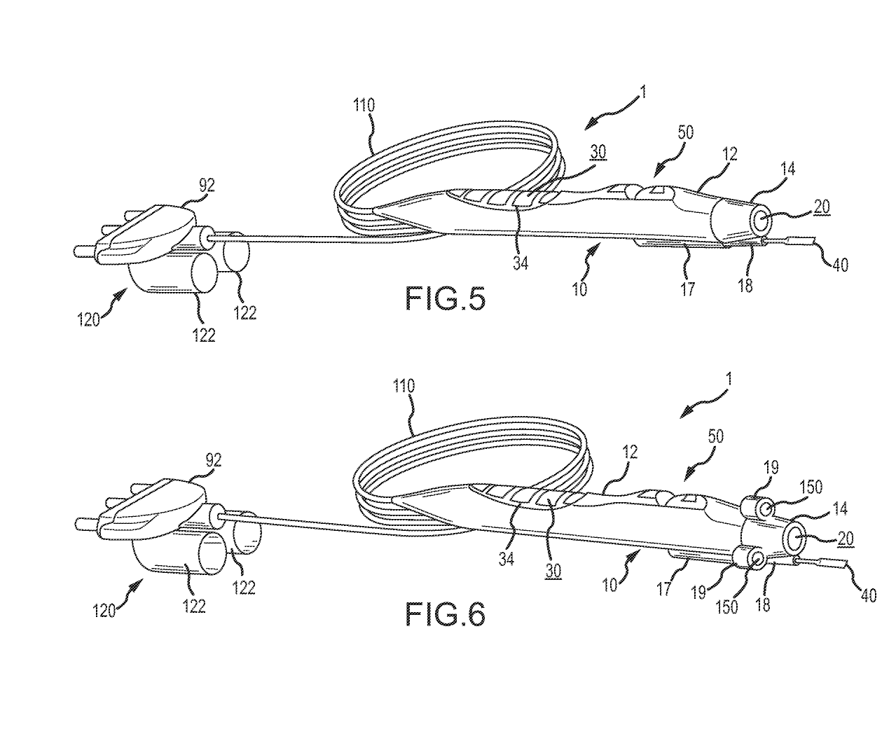

Reference is now made to FIG. 6 which illustrates the modified hand-held instrument 1 shown in FIG. 5 with the further inclusion of a plurality of light emitters_150 supportably interconnected to and outside of the second member 14 near a distal end thereof. The light emitters 150 emit light in a direction distal to the distal end of second member 140. The light emitters 150 may be supported by support hubs 19 provided on the second member 14. In one approach, the light emitters 150 may each comprise a light emitting diode (LED).

While two emitters 150 are shown in FIG. 6, it is contemplated that three emitters may be provided in spaced relation about the second member 14 (e.g. offset about 120° relative to each other in a ring-like configuration). The provision of light emitters 150 at the distal end of the hand-held instrument 1 may provide for the illumination of a volume that includes at least a portion of the electrode 40 (e.g. at least a distal end portion of electrode 40), thereby facilitating electrosurgical procedures. Further, the provision of a plurality of light emitters 150 located about the second member 14 may provide for optimal illumination of a volume by avoiding or substantially reducing any “shadowing” effects resulting from the projecting electrosurgical electrode 40 and/or the distal end of the second member 14.

The light emitters 150 may be powered and selectively turned on and off in tandem with and in the same manner as described above in relation to the powering and operation of the motor 70. In that regard, the light emitters 150 may be electrically interconnected for receiving a power signal in tandem with motor 70, as controlled by one of the switches 50. In other arrangements, switches 50 may be provided to facilitate separate on/off control of the motor 70 and light emitters 150.

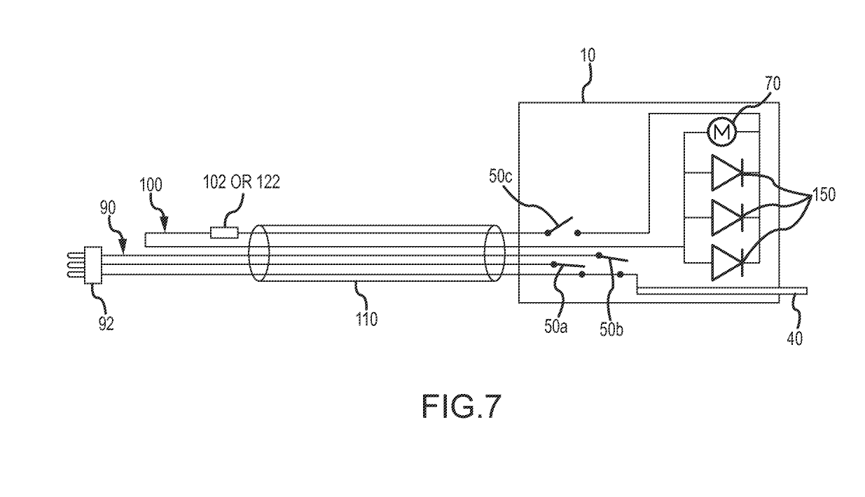

Reference is now made to the schematic illustration of FIG. 7 which illustrates electrical connections for components of the hand-held instrument 1 described hereinabove. In particular, in relation to each of the described arrangements, housing 10 houses motor 70 that may be powered via electrical cabling 100 by either use of plug-in coupler 102 (e.g. an AC/DC converter transformer) at an external energy source (e.g. a 110 Volt AC source), or by one or more batteries (e.g. to provide a 6 volt DC signal) located at battery module 122. To provide for on/off control of motor 70, switches 50 may include switch 50c at housing 10 for selective operator control.

Further, an electrosurgical electrode 40 may be supportably interconnected to housing 10 and powered via electrical cabling 90 by use of a coupler end 92 at an electrosurgical generator. The electrosurgical generator may provide tissue cutting and tissue coagulation signals via separate electrical lines comprising electric cabling 90. Selective operator control over the provision of either a tissue coagulation signal to electrode 40 or a tissue cutting signal to electrode 40 may be provided via the provision of switches 50a and 50b at housing 10.

For the hand-held instrument 1 shown in FIG. 6, light emitters 150 may be housed in housing 10 and powered in tandem with motor 70 via electrical cabling 100 by either plug-in coupler 102 or by one or more batteries located at battery module 122, as described above in relation to the powering of motor 70. To provide for on/off control of the light emitters 150, switch 50c may be utilized. In other arrangements, switches 50 may include a separate additional switch for selective on/off control of light emitters 150.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1. A hand-held instrument, comprising:
a housing sized for hand-held use and defining at least a portion of an internal volume of the hand-held instrument;
an inlet port and an outlet port for gas passage in to and out of the internal volume;
an impeller disposed within the internal volume between the inlet port and the outlet port; and,
a motor disposed within the internal volume for rotating said impeller to induce gas flow in to the inlet port and out of the outlet port.

2. A hand-held instrument as recited in claim 1, further comprising:
a filter located at the outlet port for filtering gas flowing out of the outlet port.

3. A hand-held instrument as recited in claim 1, wherein said hand-held instrument has a proximal end and a distal end, wherein said inlet port is located proximate to said distal end and distal to said impeller, and wherein said outlet port is located proximal to said impeller.

4. A hand-held instrument as recited in claim 3, further comprising:
a gas flow member disposed within the internal volume, proximal to the impeller and distal to the outlet port, for directing gas flow from the impeller.

5. A hand-held instrument as recited in claim 3, wherein said outlet port is located along a side of said housing.

6. A hand-held instrument as recited in claim 5, further comprising:
an internal sidewall defining a portion of the internal volume proximal to the impeller, wherein a portion of the internal sidewalls is angled across a longitudinal axis of the hand-held instrument to the outlet port.

7. A hand-held instrument as recited in claim 3, said housing comprising:
a first member that includes the outlet port; and,
a second member that includes the inlet port wherein the first member and the second member are interconnected and disposed for relative movement therebetween.

8. A hand-held instrument as recited in claim 7, said first and second members being disposed for relative movement therebetween along a longitudinal axis of the hand-held instrument, wherein said second member is positionable in at least a first position and a second position distal to said first position.

9. A hand-held instrument as recited in claim 8, wherein said second member is selectively positionable by a user at a continuum of positions along the longitudinal axis.

10. A hand-held instrument as recited in claim 9, wherein said first and second members are slidably interconnected along the longitudinal axis.

11. A hand-held instrument as recited in claim 10, wherein a proximal end portion of said second member extends in to a distal end portion of said first member.

12. A hand-held instrument as recited in claim 1, further comprising:
at least one light emitter supportably interconnected to said housing for emitting light in a direction distal to a distal end of said housing.

13. A hand-held instrument as recited in claim 1, further comprising:
a plurality of light emitters supportably interconnected to said housing for emitting light in a direction distal to a distal end of said housing, wherein said plurality of light emitters are located in spaced relation about the housing.

14. A hand-held instrument as recited in claim 1, further comprising:
a hand switch for controlling operation of said motor.

15. A hand-held instrument as recited in claim 14, wherein said hand switch is located along a side of said housing.

16. A hand-held instrument as recited in claim 1, wherein said motor is disposed proximal to said impeller and said outlet port.

17. A hand-held instrument as recited in claim 1, further comprising:
at least one battery for electrically powering said motor.

18. A hand-held instrument as recited in claim 18, wherein said motor and said battery are each disposed within the internal volume proximal to said impeller and said outlet port.

19. A hand-held instrument as recited in claim 17, wherein said motor is disposed within the internal volume proximal to said impeller and said outlet port, and further comprising:
electrical cabling for providing a power signal to the motor, wherein a distal end of the electrical cabling is electrically interconnected or interconnectable to the motor; and,
a battery module for supporting at least one battery in electrical contact with said electrical cabling at a location along the electrical cabling that is spaced from the housing of the hand-held instrument.

20. A hand-held instrument as recited in claim 1, comprising:
an energy emission component for energy application to a tissue site.

21. A hand-held instrument as recited in claim 20, wherein said energy emission component comprises an electrosurgical electrode supportably connected to and extending distally from a distal end of the housing.

22. A hand-held instrument as recited in claim 21, said housing comprising:
a first member that includes the outlet port; and
a second member that includes the inlet port, wherein first member and second member are interconnected and disposed for relative movement therebetween, and wherein the electrosurgical electrode is supportably connected to said second member for co-movement therewith.

23. A hand-held instrument as recited in claim 22, further comprising:
a plurality of light emitters supportably interconnected to said second member for emitting light in a direction distal to a distal end of said second member, wherein said plurality of light emitters are located in spaced relation about the second member.

24. A hand-held instrument as recited in claim 22, wherein said first and second members are slidably interconnected so that said second member is selectively positionable by a user at a continuum of positions along a longitudinal axis of the hand-held instrument; and further comprising:
a first electrical contact mounted to the first member; and
a second electrical contact mounted to the second member, so that said second electrical contact is in contact engagement with said first electrical contact member when the second member is selectively positioned by a user at any of said continuum of positions.

25-50. (canceled)
